(19) United States
(12) Patent Application Publication
(10) Pub. No.: US 2023/0358698 A1
(43) Pub. Date: Nov. 9, 2023

(54) METHOD FOR CHARACTERIZING COMPOUNDS OF INTEREST IN A MEASURING CHAMBER HAVING A VARIATION IN RELATIVE HUMIDITY

(71) Applicant: ARYBALLE, GRENOBLE (FR)

(72) Inventors: Cyril Herrier, Grenoble (FR); David Harbine, Grenoble (FR); Yanis Caritu, Grenoble (FR)

(21) Appl. No.: 17/780,635

(22) PCT Filed: Nov. 25, 2020
(86) PCT No.: PCT/EP2020/083372

§ 371 Date: May 27, 2022

(30) Foreign Application Priority Data
Nov. 29, 2019 (FR) FR1913555

(51) Int. Cl. G01N 27/12; G01N 33/00; G01N 21/552

(52) U.S. Cl. CPC G01N 27/121; G01N 33/0026; G01N 33/0006; G01N 33/0034; G01N 21/553; G01N 2201/12746

(57) ABSTRACT

A method for characterizing compounds of interest, introduced into a measuring chamber of an electronic nose, includes injecting a first gas sample formed from a carrier gas without the compounds of interest forming a second gas sample from the carrier gas with the compounds of interest; determining a measurement signal (Sₖ(tᵢ)); measuring values φ1, φ2 of the relative humidity; determining corrective parameter (\tilde{S}ₖ^{ref}|_{φ2}; ΔSₖ^{ref}|_{Δφ}); and determining a useful signal (Suₖ(tᵢϵP2)) by correcting the measurement signal associated with the second gas sample using the determined corrective parameter, and characterizing the compounds of interest based on the useful signal.

### CROSS-REFERENCE TO RELATED APPLICATIONS

This application is a national phase entry under 35 U.S.C. § 371 of International Patent Application PCT/EP2020/083372, filed Nov. 25, 2020, designating the United States of America and published as International Patent Publication WO 2021/105213 A1 on Jun. 3, 2021, which claims the benefit under Article 8 of the Patent Cooperation Treaty to French Patent Application Serial No. FR1913555, filed Nov. 29, 2019.

### TECHNICAL FIELD

The field of the disclosure is that of the characterization of compounds of interest in a gas sample located in a measuring chamber exhibiting a variation in relative humidity.

### BACKGROUND

The ability to characterize and to analyze compounds of interest odor molecules or volatile organic compounds, for example contained in gas samples is an increasingly important issue in various fields, and notably in those of health, in the food-processing industry and in the perfume industry (fragrances), and with regard to olfactory comfort in confined public or private places (motor vehicle, hotel industry, shared places, etc.), etc. The characterization of compounds of interest present in a gas sample is performed by a characterization system called an “electronic nose.”

Various characterization approaches exist, which differ from one another notably in that the compounds of interest or receptors need or do not need to be “labelled” beforehand with a marker. Unlike, for example, detection by fluorescence, which requires such markers to be used, detection by surface plasmon resonance (SPR) is a label-free technique.

The SPR characterization technique may be implemented by an electronic nose using, for example, SPR imaging technology, the compounds of interest then being contained in a gas sample and interacting through adsorption/desorption with receptors located at a plurality of distinct sensitive sites. This characterization technique consists in detecting in real tune an optical signal, associated with each of the sensitive sites, representative of the temporal variation in the local refractive index due to the adsorption/desorption interactions of the compounds of interest with the receptors.

Insofar as the chemical or physical affinity of interaction of the compounds of interest with the receptors is not known a priori, the characterization of the compounds of interest then amounts to determining a (steady-state) equilibrium value of a parameter representative of the adsorption/desorption interactions of the compounds of interest with the receptors, here representative of the temporal variation in the local refractive index for each of the sensitive sites. An interaction pattern, or a signature, is thus obtained which characterizes the compounds of interest. Specifically, the adsorption/desorption interactions of the compounds of interest at sensitive sites (functionalized surfaces) benefiting from differentiated adsorption characteristics allows the molecules present in the gas that have attached to the surface of the various sensitive sites to be determined.

In this regard, FIGS. 1A and 1B illustrate an example of an electronic nose such as described in patent application WO2018/158458. This type of electronic nose 1 generally comprises a fluid-supplying device 2, a device 3 for measuring by SPR imaging (also referred to herein as an “optical measuring device” 3), and a processing unit (not shown).

The optical measuring device 3 comprises a measuring chamber 4 intended to receive the gas sample, in which chamber is located a measurement carrier 5 on which is located a matrix-array of sensitive sites 6ₖ. The measurement carrier 5 is formed from a metal layer to which are fastened various receptors suitable for interacting with the compounds of interest, the various receptors being arranged so as to form sensitive sites 6ₖ that are distinct from one another. These receptors are then located at the interface between the metal layer and a dielectric medium, here a gaseous medium.

This optical measuring device 3 further comprises a light source 7 for emitting an excitation optical signal and an image sensor 8. At least one focusing or collimating lens and at least one polarizer may be provided on the optical path between the light source 7 and the image sensor 8, in known manner. The light source 7 is designed to emit the excitation optical signal in the direction of the measurement carrier 5, at a working angle θ_{R} allowing surface plasmons to be generated thereon. The reflected portion of the excitation optical signal, forming a measurement optical signal, is then detected by the image sensor 8. The intensity of the optical measurement signal depends locally on the refractive index of the measurement carrier 5, which itself depends on the surface plasmons generated and on the amount of material located at each sensitive site 6ₖ, this amount of material varying over time depending on the interactions between the sensitive compounds and the receptors.

The processing unit of the electronic nose is suitable for analyzing “sensorgrams,” i.e., the signals corresponding to the time evolution of the parameter representative of the adsorption/desorption interactions of the compounds of interest with the receptors of each of the various sensitive sites 6ₖ, with the aim of extracting therefrom information on the kinetics of the interaction (adsorption and desorption) of the compounds of interest with the receptors. These sensorgrams may be measurement signals Sₖ(t) corresponding to the intensity of the measurement signal detected in real time by the image sensor 8 of each of the sensitive sites 6ₖ, or be “useful” signals Suₖ(t) corresponding to the time evolution of the variation Δ % Rₖ(t) of the reflectivity associated with each of the sensitive sites 6ₖ. The reflectivity % R is defined as the ratio of the intensity of the measurement optical signal detected by the image sensor 8 to the intensity of the excitation optical signal emitted by the light source 7. The variation in reflectivity Δ % R is obtained by subtracting from the time evolution of the reflectivity % R(t) a reference value (baseline) associated with just the gas present inside the measuring chamber, independently of the compounds of interest.

The useful signals Suₖ(t), therefore, have the same steady-state initial value, preferably substantially equal to zero, before the introduction of the compounds of interest into the measuring chamber 4. Thus, this reference value (baseline) which expresses the effect of just the gas (without the compounds of interest) on each of the sensitive sites 6ₖ is subtracted from the corresponding measurement signal Sₖ(t). The intensity of the useful signals Suₖ(t) then evidences the addition of the compounds of interest to the measuring chamber 4.

Finally, the fluid-supplying device 2 is suitable for introducing the compounds of interest into the measuring chamber 4, under conditions that allow analysis of the sensorgrams and, therefore, characterization of the compounds of interest. In this regard, the article by Brenet et al. entitled Highly-Selective Optoelectronic Nose based on Surface Plasmon Resonance Imaging for Sensing Gas Phase Volatile Organic Compounds, Anal. Chem. 2018, 90, 16, 9879-9887, describes a method for characterizing a gas sample using an SPR imaging electronic nose.

The characterization method consists in supplying the measuring chamber with a gas sample in such a way that the kinetics of the interaction between the compounds of interest and the receptors reaches a steady equilibrium state. More specifically, the fluid injection step successively comprises:
a first, “initial” phase P1, in which just a reference gas, without the compounds of interest, is injected into the measuring chamber. This reference gas is generally identical to the carrier gas of the gas sample
a second, “characterization” phase P2, in which the gas sample, formed of the carrier gas and the compounds of interest, is injected into the measuring chamber;
a third, “dissociation” phase P3, in which the compounds of interest are removed from the measuring chamber.

The initial phase P1 allows the acquisition of the reference value (baseline) mentioned above which is then to be subtracted from the measurement signals Sₖ(t) in order to obtain useful signals Suₖ(t) (in other words the time evolution of the variation in reflectivity. Δ % Rₖ(t) for each sensitive site). As mentioned above, this fluid injection phase is carried out such that the sensorgrams reveal the presence of a transient assimilation state followed by a steady equilibrium state. When this steady equilibrium state is reached, the (steady) equilibrium values of the useful signals Suₖ(t) are extracted by the processing unit, and define the signature of the compounds of interest.

However, it appears that the relative humidity inside the measuring chamber has an effect on the intensity of the optical measurement signal, as indicated in the article by Shao et al. entitled Mechanism and Characteristics of Humidity Sensing with Polyvinyl Alcohol-Coated Fiber Surface Plasmon Resonance Sensor, Sensors 2018, 18, 2029. In this article, the authors use an SPR sensor as a humidity sensor. However, in the context of a method for characterizing compounds of interest using an electronic nose, the variation in relative humidity in the measuring chamber forms measurement noise which negatively affects the quality of the characterization. In addition, in the case of the relative humidity varying over long time periods and, therefore, varying from one characterization to the next for the same compounds of interest and the same operating conditions, this results in a temporal drift which makes the signatures of these same compounds of interest different from one another.

### BRIEF SUMMARY

The object of embodiments of the disclosure is to at least partially remedy the drawbacks of the prior art and, more particularly, to provide a method for characterizing compounds of interest which limits or even eliminates the measurement noise due to the difference in relative humidity in the measuring chamber between the initial phase P1 and the characterization phase P2.

To that end, the subject of the disclosure is a method for characterizing compounds of interest introduced into a measuring chamber of an electronic nose comprising at least one sensitive site having receptors with which the compounds of interest are able to interact through adsorption/desorption, the method comprising the following steps:
injecting, into the measuring chamber: in a first phase P1, a first gas sample formed of a carrier gas without the compounds of interest, and then in a second phase P2, a second gas sample formed of at least the carrier gas and of the compounds of interest;
determining, in phases P1 and P2, a measurement signal representative of the interactions between the gas sample present and the receptors of the sensitive site 6ₖ, at various measurement times tᵢ, in response to an excitation signal issued at the sensitive site.

According to embodiments of the disclosure, the method comprises the following steps:
a value φ1 of the relative humidity φ in the measuring chamber in the first phase P1, and a value φ2 in the second phase P2, φ2 being different from φ1;
determining a corrective parameter associated with the sensitive site (6ₖ), based on: at least the measured value φ2 of the relative humidity, and a predetermined calibration function associated with the sensitive site, the calibration function expressing a variation in a parameter representative of the measurement signal associated with the first gas sample as a function of the relative humidity; and
determining a useful signal by correcting the measurement signal associated with the second gas sample based on at least the determined corrective parameter, and characterizing the compounds of interest based on the useful signal.

The following are some preferred but non-limiting aspects of this characterization method.

The method may comprise a phase of determining the calibration function denoted by hₖ comprising the following steps:
injecting the first gas sample into the measuring chamber such that the relative humidity φ varies progressively, and measuring the relative humidity φ;
determining a measurement signal Sₖ(tᵢ) in the injection step, and then determining a reference value \tilde{S}ₖ^{ref} based on the determined measurement signal \tilde{S}ₖ(tᵢ) and for each measured relative humidity value φ; and
determining a calibration function hₖ expressing the variation in the reference value \tilde{S}ₖ^{ref} as a function of the relative humidity φ, based on the determined reference values \tilde{S}ₖ^{ref} and the measured relative humidity values φ.

The corrective parameter may be a reference value \tilde{S}ₖ^{ref}|_{φ2} representative of the measurement signal Sₖ(tᵢ) of the first gas sample for the measured relative humidity φ2.

The step of determining the useful signal may comprise the following sub-steps:
determining a reference value \tilde{S}ₖ^{ref}|_{φ1} representative of the measurement signal of the first gas sample for the measured relative humidity φ1, based on the calibration function hₖ;
determining a useful signal Suₖ(tᵢϵP1) associated with the first sample by subtracting the determined reference value from the measurement signal Sₖ(tᵢϵP1) determined in the initial phase P1;
determining a reference value Suₖ^{ref} based on the determined useful signal Suₖ(tᵢϵP1) associated with the first sample; and
determining a corrected useful signal Sucₖ(tᵢϵP2) associated with the second gas sample by subtracting the determined reference value Suₖ^{ef} from the useful signal Suₖ(tᵢϵP2); The compounds of interest may be characterized (150) based on the corrected useful signal Sucₖ(tᵢϵP2) associated with the second gas sample.

The method may comprise a phase of determining the calibration function denoted by fₖ comprising the following steps:
injecting the first gas sample into the measuring chamber such that multiple injection cycles are carried out, each cycle comprising a first injection of the first gas sample at a relative humidity φ1 followed by a second injection of the first gas sample at a relative humidity φ2 different from φ1, and determining the difference in relative humidity Δφ between φ1 and φ2 varying from one cycle to the next;
determining a measurement signal \tilde{S}ₖ(tᵢ) in the various injection cycles, and determining a difference in reference values Δ\tilde{S}ₖ^{ref} for each injection cycle and for each determined difference in relative humidity Δφ, based on the measurement signal Śₖ(tᵢ) determined in the various injection cycles; and
determining the calibration function fₖ expressing the variation in the difference in reference value Δ\tilde{S}ₖ^{ref} as a function of the difference in relative humidity Δφ, based on the determined values of the difference in reference value Δ\tilde{S}ₖ^{ref} and the determined values of the difference in relative humidity Δφ.

The corrective parameter may be the sum of a value of the difference in reference values Δ\tilde{S}ₖ^{ref} for the difference in relative humidity Δφ determined based on the relative humidity, measured in the first phase P1 and in the second phase P2, and a reference value Sₖ^{ref}|_{φ1} of associated with the first gas sample and determined based on the measurement signal Sₖ(tᵢϵP1) determined in the first phase P1.

The step of determining the useful signal may comprise subtracting the corrective parameter from the measurement signal Sₖ(tᵢϵP2) associated with the second gas sample and determined in the second phase P2.

The calibration function may be a polynomial, logarithmic, or exponential function. It may be a second-degree polynomial function.

The electronic nose may comprise a device for measuring the interactions between the compounds of interest and the surface plasmon resonance optical receptors. As a variant, it may comprise a device for measuring the interactions between the compounds of interest and the resistive, piezoelectric, mechanical, acoustic or optical receptors.

### BRIEF DESCRIPTION OF THE DRAWINGS

Other aspects, aims, advantages and features of the disclosure will become more clearly apparent on reading the following detailed description of preferred embodiments thereof, this description being given by way of non-limiting example and with reference to the appended drawings, in which:

FIGS. 1A and 1B, which have already been described, are schematic and partial views, in cross section (FIG. 1A) and seen from above (FIG. 1B), of an SPR imaging electronic nose according to one example of the prior art and of the sensitive sites of the measurement carrier;

FIG. 1C is a schematic partial view of an SPR imaging electronic nose according to one embodiment;

FIG. 2 is an example of sensorgrams Suₖ(t) measured by the electronic nose according to the example of the prior art, these sensorgrams corresponding here to the time evolution of the variation in the reflectivity Δ % Rₖ(t) associated with each of the sensitive sites 6ₖ;

FIG. 3A is an example of three interaction patterns (signatures) obtained using a characterization method according to the prior art, showing the deterioration in the characterization of compounds of interest due to a difference in relative humidity in the measuring chamber between the initial phase P1 and the characterization phase P2;

FIG. 3B is an example of various measurement signals Sₖ(t) obtained using an SPR imaging electronic nose for various relative humidity φ situations in the measuring chamber: i.e., in the case where the relative humidity is constant and equal to φ1, in the case of the relative humidity being constant and equal to φ2, different from φ1, and in the case of the relative humidity, going from φ1 to φ2 between the initial phase P1 and the characterization phase P2;

FIG. 4 is a flowchart of a characterization method according to a first embodiment;

FIG. 5 is an example of a calibration function ilk expressing the change in a reference value \tilde{S}ₖ^{ref} associated with a first gas sample (carrier gas without the compounds of interest) as a function of the relative humidity φ;

FIG. 6 illustrates the three interaction patterns (signatures) already illustrated in FIG. 3A, and an interaction pattern obtained using the characterization method according to the first embodiment;

FIG. 7 is a flowchart of a characterization method according to a second embodiment;

FIG. 8A is an example of a difference ΔSₖ(t) between measurement signals Sₖ(t) for various injection cycles injecting a first gas sample (carrier gas without the compounds of interest), each cycle comprising injection of the first gas sample with constant relative humidity φ1 over the cycles, and injection of the same first gas sample with varying relative humidity φ2 over the cycles;

FIG. 8B illustrates the difference in relative humidity Δφ over the cycles illustrated in FIG. 8A; and

FIG. 8C is an example of a calibration function hₖ expressing the change in the difference in reference value Δ\tilde{S}ₖ^{ref} as a function of the difference in relative humidity Δφ.

### DETAILED DESCRIPTION

In the figures and in the remainder of the description, the same references have been used to designate identical or similar elements. In addition, the various elements have not been shown to scale for the sake of clarity of the figures. Moreover, the various embodiments and variants are not mutually exclusive and may be combined with one another: Unless indicated otherwise, the terms “substantially,” “about” and “of” the order of mean to within 10%, and preferably to within 5%. Moreover, the terms “comprised between . . . and . . . ” and equivalents mean inclusive of limits, unless indicated otherwise.

The disclosure relates to the characterization of compounds of interest present in a carrier gas forming a gas sample to be analyzed. Characterization is carried out using an analysis system called an “electronic nose,” which comprises: a measuring device; a, fluid-supplying device; a humidity sensor; and a processing unit. As detailed below, the measuring device comprises a measuring chamber that is suitable for receiving a gas sample, in which measuring chamber there is at least one sensitive site, and preferably a plurality of distinct sensitive sites, the one or more sensitive sites each having receptors suitable for interacting, by adsorption/desorption, with the compounds of interest.

By way of illustration, the electronic nose uses surface plasmon resonance (SFR) measurement technology. The measuring device then comprises an optical sensor which may be an image sensor, the measuring chamber then having a plurality of sensitive sites 6ₖ (k being the rank of the sensitive site in question), or which may be a photodetector, in which case the measurement is carried out by searching for the minimum angle of reflection, this angle being representative of the variation in index. As a variant, other measurement technologies may be implemented, such as measurement using MEMS or NEMS electromagnetic resonators. More broadly, the measuring device may be a resistive, piezoelectric, mechanical, acoustic or optical measuring device.

Generally, what is meant by “characterization” is obtaining information representative of the interactions of the compounds of interest contained in the gas sample with the receptors of the one or more sensitive sites of the electronic nose. The interactions in question are here events resulting in the compounds of interest adsorbing on and/or desorbing, from the receptors. This information thus forms an interaction pattern, or in other words a “signature” of the compounds of interest, this pattern being representable, for example, in the form of a histogram or of a radar chart. More precisely, in the case where the electronic nose comprises N distinct sensitive sites, the interaction pattern is formed by N representative items of information, these being taken from the measurement signal obtained for the sensitive site 6ₖ in question.

Generally, the compounds of interest (analytes) are elements intended to be characterized by the electronic nose, and contained in a gas sample. They may be, by way of illustration, bacteria, viruses, proteins, lipids, volatile organic molecules, inorganic compounds, inter alia. Moreover, the receptors (ligands) are elements that are fastened to the sensitive sites and that exhibit a capacity for interaction with the compounds of interest, though the chemical and/or physical affinities between the sensitive compounds and the receptors are not necessarily known. The receptors of the various sensitive sites preferably have different physicochemical properties, which have an impact on their ability to interact with the compounds of interest. It may be a question, by way of example, of amino acids, peptides, nucleotides, polypeptides, proteins, organic polymers, inter atria.

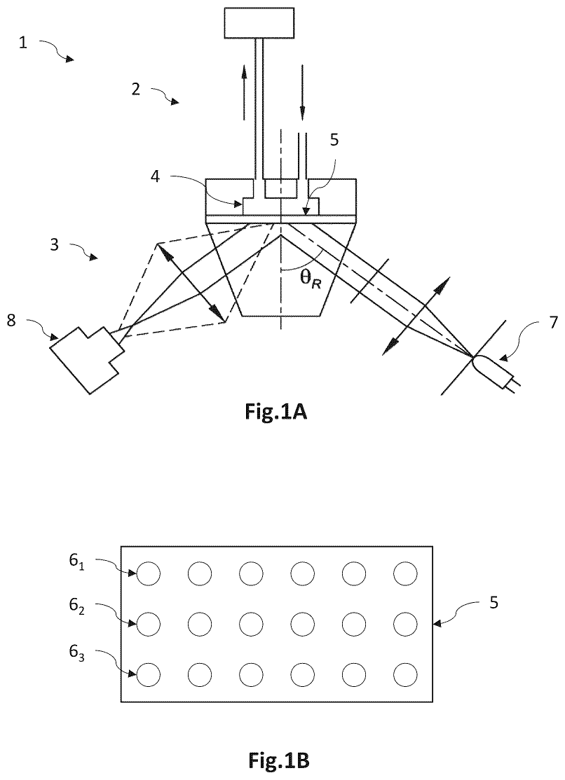

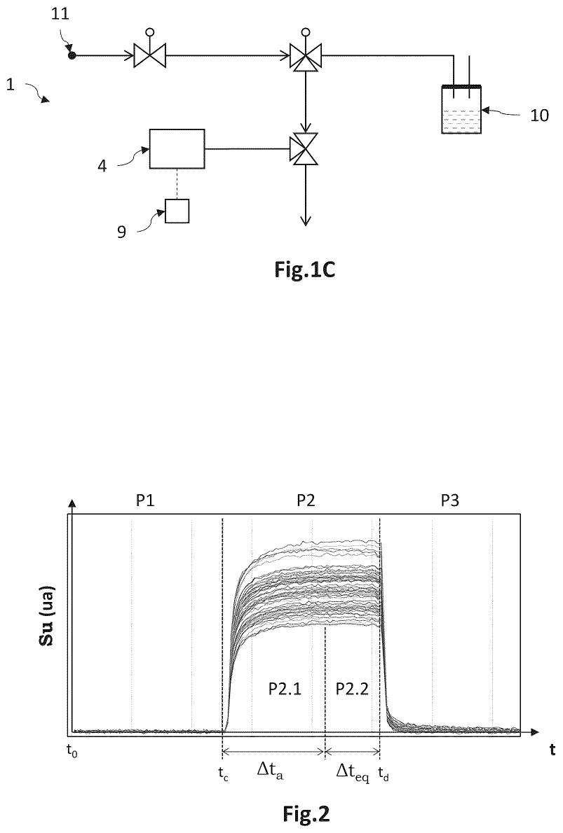

With reference to FIGS. 1A to 1C, the electronic nose 1 is here an optoelectronic system allowing compounds of interest, for example odor molecules or volatile organic compounds inter alia, contained in a gas sample introduced into a measuring chamber 4 of the electronic nose, to be characterized. The electronic nose 1 shown in these figures is based here on SPR technology and has, in this example, the features of the Kretschmann configuration, which is known to those skilled in the art, though the disclosure is not, however, limited to this configuration. However, as mentioned above, other measurement techniques may be used, such as measurements of the resonant frequency of a MEMS or NEMS microresonator that is functionalized so that it has at least one sensitive site equipped with receptors.

The electronic nose 1 comprises a plurality of sensitive sites 6ₖ distinct from one another and located in a measuring chamber 4 which is intended to receive the gas sample to be analyzed, these sensitive sites each being formed of receptors capable of interacting with the compounds of interest to be studied (see FIG. 1B). The sensitive sites 6ₖ are distinct from one another in the sense that they comprise receptors that are different, in terms of chemical or physical affinity with respect to the compounds of interest to be analyzed, and are, therefore, intended to deliver interaction information that differs from one sensitive site 6ₖ to the next. The sensitive sites 6ₖ are distinct regions of a measurement carrier 5, and may be contiguous or spaced apart from one another. The electronic nose 1 may further comprise a plurality of identical sensitive sites 6ₖ, for example with the aim of detecting any measurement drift and/or of allowing the identification of a defective sensitive site.

The electronic nose comprises a measuring device 3 (also referred to herein as an “optical measuring device” 3), here an SPR imaging device, allowing, for each sensitive site 6ₖ, the interactions of the compounds of interest with the receptors to be quantified, here via measurement in real time of the intensity of a measurement optical signal from the sensitive site 6ₖ in question, this optical signal being a portion, here a reflected portion, of an excitation optical signal emitted by a light source 7. The intensity of the optical measurement signal detected by the optical sensor 8 (also referred to herein as the “image sensor” 8) is directly correlated, in particular, with the adsorption/desorption interactions of the compounds of interest with the receptors. In the case of techniques for measuring the resonant frequency of a NEMS or MEMS microresonator, the measurement signal may be an electrical signal representative of the vibration of a microbeam or the like.

In the context of measurement by SPR imaging, the measuring device 3 is suitable for acquiring, in real time, the measurement optical signal from all of the sensitive sites 6ₖ. Thus, the measurement optical signals issued from the sensitive sites 6ₖ in response to the excitation optical signal are detected together and in real time, in the form of an image acquired by the same optical sensor 8.

Thus, the optical measuring device 3 comprises a light source 7 suitable for transmitting an excitation optical signal in the direction of the sensitive sites 6ₖ, and for generating surface plasmons on the measurement carrier 5. The light source 7 may be formed from a light-emitting diode, the emission spectrum of which has an emission peak centered on a central wavelength λ_{c}. Various optical elements (lenses, polarizer, etc.) may be placed between the light source 7 and the measurement carrier 5.

The optical measuring device 3 further comprises an image sensor 8, and here an image sensor, i.e., a matrix-array optical sensor suitable for collecting or detecting an image of the optical signal issued from the sensitive sites in response to the excitation optical signal. The image sensor 8 is a matrix-array photodetector, a CMOS or CCD sensor for example. It therefore comprises a matrix-array of pixels whose spatial resolution is such that, preferably, a plurality of pixels acquires the measurement optical signal issued from a given sensitive site 6ₖ.

The processing unit (not shown) allows the processing operations described below in the context of the characterization method to be implemented. It may comprise at least one microprocessor and at least one memory. It is connected to the optical measuring device 3, and more precisely to the image sensor 8. It comprises a programmable processor able to execute instructions stored on a data storage medium. It further comprises at least one memory containing the instructions required to implement the characterization method. The memory is also suitable for storing the information computed at each measurement time.

As described below, the processing unit is notably suitable for storing and processing a plurality of images, called elementary images, acquired at a given sampling frequency f_{c} in a measurement period Δt, in order to determine, at the current time tᵢ, a measurement signal Sₖ(tᵢ) associated with the sensitive site 6ₖ. Preferably, the measurement signal Sₖ(tᵢ) corresponds, at a measurement time to the average of the intensity of the optical signal reflected and detected by the image sensor 8 on the pixels associated with the sensitive site 6ₖ. The average of the optical intensity detected on the pixels may be made for one or more images of the sensitive site 6ₖ, as described in detail below.

The fluid-supplying device 2 is designed to supply the measuring chamber 4 with a first gas sample formed of just the carrier gas (i.e., without the compounds of interest) in the initial phase P1, and with a second gas sample formed of the gas carrier and the compounds of interest in the characterization phase P2. The second gas sample differs from the first sample primarily in that it contains the compounds of interest. One or more additional gases may be present, but they are odorless and so they provoke substantially no response from the electronic nose 1. An example of additional gas present in the second gas sample may be the diluent in the vapor phase. As described with reference to FIG. 1C, the compounds of interest may be stored in a liquid diluent contained in a reservoir 10. The vapor phase of the diluent and the compounds of interest are added to the carrier gas (e.g., humid air) to form the second gas sample. The first and second gas samples differ from one another here also in their relative humidity value.

For this, as illustrated in FIG. 1C, the fluid-supplying device 2 comprises an inlet 11 for carrier gas, and a reservoir 10 for compounds of interest. Here, the reservoir 10 contains a diluent in which the compounds of interest are found. It comprises multiple fluid lines which connect the inlet 11 for the carrier gas and the reservoir 10, on the one hand, to the inlet of the measuring chamber 4, on the other hand, and comprises valves and possibly mass flow regulators. It thus allows the measuring chamber 4 to be supplied with the first gas sample (for example, humid air at relative humidity φ1) in the initial phase P1 and in the dissociation phase P3, and with the second gas sample (e.g., humid air at relative humidity φ2, compounds of interest, and diluent in the vapor phase) in the characterization phase P2. It may be able to ensure that the concentration of the compounds of interest in the measuring chamber remains constant over time. In addition, the electronic nose according to one embodiment further comprises a humidity sensor 9 able to measure the relative humidity in the measuring chamber. The humidity sensor 9 may be arranged in the measuring chamber, or upstream or downstream thereof. It is connected to the processing unit, which may also be designed to calculate a difference in relative humidity, between the initial phase P1 and the characterization phase P2.

FIG. 2 illustrates an example of sensorgrams Suₖ(t) associated with sensitive sites 6ₖ of an SPR imaging electronic nose, in the context of a characterization method in which the sensorgrams each have a profile that is said to be conventional, i.e., they reveal the presence of an equilibrium (i.e., steady) state between the compounds of interest and the receptors. In this example, the relative humidity φ does not vary significantly between the initial phase P1 and the characterization phase P2.

A sensorgram Suₖ(t) corresponds to the time evolution of a characterization parameter representative of the interactions between the compounds of interest and the receptors of a sensitive site 6ₖ in question. It is determined based on the intensity of the measurement signal Sₖ(t) issued from sensitive site 6ₖ in response to the emission of an excitation signal. In this example, the characterization parameter is the variation Δ % Rₖ(t) relative to a reference value (baseline) of the reflectivity % Rₖ(t) associated with the sensitive site 6ₖ, but it may be, in another configuration of the electronic nose, the variation in the transmission coefficient. The variation in the reflectivity Δ % Rₖ(t) is here correlated with the change in the refractive index of the sensitive site 6ₖ in question, which is dependent on the adsorption and desorption interactions of the compounds of interest with the receptors of the sensitive site 6ₖ.

In a known manner, a conventional-profile sensorgram Suₖ(t) exhibits an initial phase P1, a phase P2 of characterizing the compounds of interest, and then a dissociating phase P3. The y-axis value of the sensorgram Suₖ(t) is notably proportional to the number of receptors of the sensitive site 6ₖ in question.

The initial phase P1 corresponds to the introduction into the measuring chamber, from time t₀ to time t_{c}, of the first sample (carrier gas not containing the compounds of interest). The measurement signals Sₖ(t), in other words the time evolution of the reflectivity % R(t) determined for each sensitive site 6ₖ between t₀≤t<t_{c}, characterize the environment in the measuring chamber for each of the sensitive sites 6ₖ. A reference value Sₖ^{ref} (baseline), generally, different from one sensitive site 6ₖ to the next, is then deduced therefrom, which is then subtracted from the measurement signal Sₖ(t) to obtain the useful signal Suₖ(t). Thus, the sensorgrams illustrate the useful signals Suₖ(t), which therefore have, in the initial phase P1, the same initial value close to zero for all of the sensitive sites 6ₖ.

The injection phase P2 corresponds to the introduction into the measuring chamber, from time t_{c} to time t_{d}, of the second gas sample (carrier gas and compounds of interest, and optionally an odorless diluent in the gas phase). This phase comprises a transient assimilation state P2.1 followed by a steady equilibrium state P2.2. In these examples, the relative humidity φ is constant over the various phases P1, P2 and P3.

The transient assimilation state P2.1 corresponds to the gradual but exponential increase (Langmuir's law) in the interactions between the compounds of interest and the receptors, as the compounds of interest are injected into the measuring chamber. The exponential growth of the sensorgrams in the assimilation state is due to the fact that there are then many more adsorption events than desorption events.

It will be noted that, in this regard, the interaction between a compound of interest A (analyte) and a receptor L (ligand) is a reversible effect characterized by a constant kₐ (in mol⁻¹·s⁻¹) of adsorption of the compound of interest A on the receptor L to form a compound of interest/receptor LA (ligand-analyte), and by a constant k_{b} (in s⁻¹) of desorption corresponding to the dissociation of the compound LA. The ratio k_{d}/kₐ forms the equilibrium dissociation constant k_{D} (in mol) that gives the value of the concentration c_{A} of compounds of interest A allowing 50% of the receptors L to be saturated.

The steady equilibrium state P2.2 is reached when the concentration c_{LA}(t) in compounds LA remains constant dc_{LA}/dt=0, i.e., when the product of the constant kₐ and the concentrations of compounds of interest c_{A}(t) and of receptors c_{L}(t) (number of adsorption events) is equal to the product of the constant IQ and the concentration c_{LA}(t) of compounds LA (number of desorption events), or in other words when the following rate equation is respected dc_{LA}/dt=kₐ×c_{A}×c_{L}−k_{d}×c_{LA}=0. The maximum steady-state value of the measurement signal is proportional to the concentration c_{A}(t) of compounds of interest A Saturation of the receptors L at the sensitive site may be achieved when the concentration c_{A} of compounds of interest A is sufficient.

The dissociating phase P3 corresponds to a step of removing the compounds of interest present in the measuring chamber, from time t_{d}, so that the concentration of compounds LA decreases, usually exponentially. This may involve introducing the first gas sample into the measuring chamber again.

However, it appears that the variation in relative humidity within the measuring chamber in the fluid injection step forms measurement noise which negatively affects the quality of the characterization. This measurement noise is noise due to a non-zero difference in relative humidity within the measuring chamber between the initial phase P1 and the characterization phase P2. It is measurement noise insofar as it results from a time evolution of a parameter (here the relative humidity) characterizing the environment inside the measuring chamber and which should theoretically remain steady over time.

In other words, the relative humidity may have a first value φ1 which is substantially constant in the initial phase P1, and a second value φ2 which is substantially constant but different from the value φ1 in the characterization phase P2. The difference in relative humidity Δφ is defined here as being equal to φ2-φ1. What is meant by relative humidity φ is the water vapor content of the gas present in the measuring chamber, and here of the carrier gas. It is the ratio of the partial pressure of the water vapor contained in the gas present to the saturation vapor pressure at the same temperature.

This measurement noise is present, in particular, when the second gas sample has a relative humidity φ2 that is different, for example lower, than that φ1 of the first gas sample. Thus, in the initial phase P1, the first gas sample may be humid air of relative humidity φ1 from the environment of the electronic nose. In the characterization phase P2, the second gas sample is formed of humid air from, for example, the environment of the electronic nose, as well as compounds of interest from the reservoir 10. However, the relative humidity φ2 of the second gas sample may be different from φ1. This is because the relative humidity of the humid air from the environment may have changed. Another possibility explaining the variation in relative humidity from φ1 to φ2 may be due to the relative humidity of the gas present in the headspace of the reservoir 10. Specifically, to form the second gas sample, the humid air at φ1 from the inlet 11 for carrier gas is introduced into the reservoir 10 and mixed with the gas present (diluent in the gas phase and compounds of interest). However, the diluent in the liquid phase may be hydrophilic, so that it may then lead to a decrease in the relative humidity φ of the humid air introduced into the headspace of the reservoir 10. Consequently, the second gas sample will have a relative humidity φ2 lower than (pi.

It should be noted that a thermodynamic equilibrium may gradually be established in the headspace of the reservoir 10, so that the hydrophilic diluent in the liquid phase will no longer cause a decrease in the relative humidity φ1 of the humid air introduced into the headspace. Thus, in this case, the second sample will gradually exhibit a relative humidity φ2 substantially equal to φ1, so that the measurement noise associated with the non-zero difference Δφ may exhibit an intensity that decreases over time. Be that as it may, the various successive characterizations will result in signatures that will then not be identical over time (temporal drift in the signatures).

This problem of measurement noise due to Δφ is particularly significant when the characterization method is carried out based on useful signals Suₖ(t), i.e., when it comprises a step of subtracting the reference value Sₖ^{ref} (baseline) from the corresponding measurement signal Sₖ(t). Specifically, the aim of this step is to exclude from the characterization of the compounds of interest the effect associated with their environment and, in particular, the effect of the carrier gas. However, it appears that while this reference value Sₖ^{ref} is representative of the carrier gas in the initial phase P1, it is no longer necessarily representative of the carrier gas in the characterization phase P2 since the physical properties of this carrier gas in the measuring chamber may have changed (variation in the relative humidity).

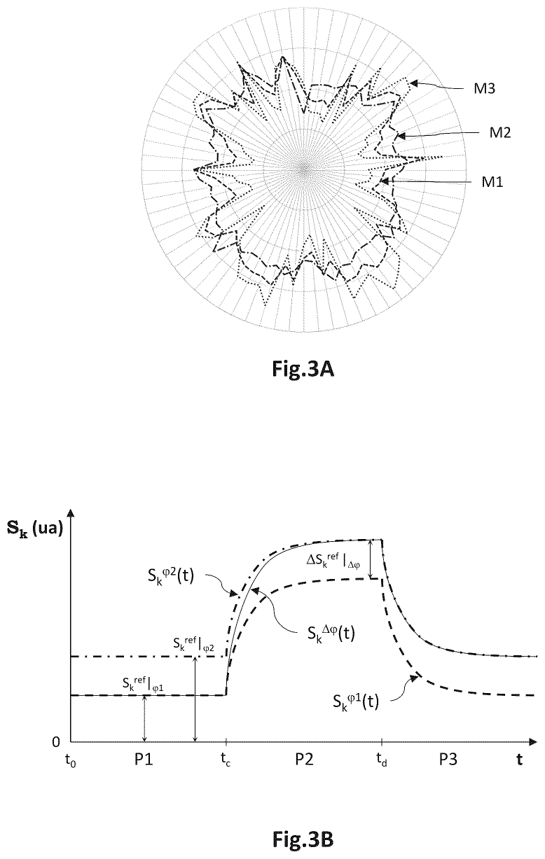

FIG. 3A illustrates three interaction patterns, or signatures, expressing the characterization of various gas samples, this characterization being performed using a characterization method according to an example from the prior art. These interaction patterns M1, M2 and M3 are here representations in the form of a radar chart of the (steady) equilibrium values determined based on the sensorgrams Suₖ(t) in the steady equilibrium state P2.2. They allow the effect of the difference in relative humidity Δφ on the characterization of the compounds of interest to be shown. To obtain these interaction patterns M1, M2, M3, the carrier gas is identical for the three tests and corresponds to humid air with an initial relative humidity φ1 of approximately 12%.

A first signature M1 corresponds to a gas sample formed of humid air with a relative humidity φ2 equal to approximately 50% and whose compounds of interest are butanol molecules. The implementation of the characterization method thus exhibits a relatively large variation in the relative humidity in the measuring chamber, which here ranges from φ1 equal to approximately 12% in the initial phase P1 to φ2 equal to approximately 50% in the characterization phase. Thus, the reference value Sₖ^{ref} is determined for a first gas sample (humid air at φ1 of 12%) and the equilibrium value is determined for the second gas sample (humid air at φ2 of 50% with the compounds of interest) by subtracting this reference value Sₖ^{ref}. This difference in relative humidity Δφ thus forms measurement noise, the effect of which has to be limited so that the interaction pattern M1 is effectively representative only of the butanol molecules.

A second signature M2 corresponds to a second gas sample formed of humid air with a relative humidity φ2 substantially equal to φ1 (i.e., 12%), and whose compounds of interest are also butanol molecules. The implementation of the characterization method makes it possible, by subtracting the reference value Sₖ^{ref} associated with the first gas sample (humid air at TO, and insofar as the variation in relative humidity Δφ is zero, to exclude the effect of the gas environment in order thus to characterize just the interactions of the compounds of interest with the receptors. Thus, the signature M2 is representative of just the compounds of interest since there is no measurement noise associated with a variation in relative humidity Δφ. It is noted that the signature M1 does not overlap with the signature M2, showing the presence of the measurement noise associated with Δφ in the case of M1. It is, therefore, important to be able to correct the signature M1 so as to tend toward the signature M2, which is the only representative of the compounds of interest, even though there is a difference in relative humidity Δφ in the measuring chamber between the initial phase P1 and the characterization phase P2.

The third signature M3 corresponds to a gas sample formed only of humid air with a relative humidity φ2 equal to approximately 50%. Here, there is measured just the effect of the variation in relative humidity Δφ on the characterization of the humid air by the electronic nose, in the absence of compounds of interest. It appears that the increase in relative humidity Δφ between the initial phase P1 and the characterization phase P2 coincides with an increase in the variation in the reflectivity Δ% Rₖ of the sensitive sites 6ₖ. It is noted that the signature M1 (humid air with non-zero Δφ and compounds of interest) is situated between the signature M2 (humid air with zero Δφ and compounds of interest) and the signature M3 (humid air with non-zero Δφ without compounds of interest), clearly showing the effect of the measurement noise associated with the non-zero difference in relative humidity Δφ on the signature of the compounds of interest. It is, therefore, important to be able to limit or even eliminate this measurement noise in order to improve the quality of the characterization of the compounds of interest.

FIG. 3B illustrates examples of measurement signals Sₖ(t) for various gas samples, thus also showing the effect of the measurement noise associated with the variation in relative humidity Δφ, on the characterization of the compounds of interest. In these examples, the carrier gas is humid air.

The measurement signal Sₖ^{φ1}(t) corresponds to the case where the relative humidity in the measuring chamber remains constant and equal to φ1 in the initial phase P1 and in the characterization phase P2. The variation in relative humidity Δφ is then zero. It has a non-zero reference value Sₖ^{ref}|_{φ1}, which corresponds to the response of the electronic nose when the first gas sample (humid air at φ1 without compounds of interest) is in the measuring chamber.

The measurement signal Sₖ^{φ2}(t) corresponds to the case where the relative humidity in the measuring chamber remains constant and equal to φ2 in the initial phase P1 and in the characterization phase P2. The variation in relative humidity Δφ is then zero. The relative humidity φ2 is here higher than φ1. It has a reference value Sₖ^{ref}|_{φ2} which is non-zero and different from Sₖ^{ref}|_{φ1}, which corresponds to the response of the electronic nose when the first gas sample (humid air at φ2 without compounds of interest) is in the measuring chamber.

The measurement signal Sₖ^{Δφ}(t) corresponds to the case where the relative humidity in the measuring chamber is not constant, and goes from the value φ1 in the initial phase P1 to the value φ2 in the characterization phase P2. The variation in relative humidity Δφ is then non-zero, and here positive. The signal exhibits the same reference value Sₖ^{ref}|_{φ1} as for the measurement signal Sₖ^{φ1}(t) in the case of the gas sample exhibiting the relative humidity φ1 in the initial phase P1. However, it exhibits the same equilibrium value as the measurement signal Sₖ^{φ2}(t) in the case of the gas sample exhibiting the relative humidity φ2 in the characterization phase P2. Thus, the measurement signal Sₖ^{Δφ}(t) gradually changes from the measurement signal Sₖ^{φ1}(t) in the initial phase P1 to the measurement signal Sₖ^{φ2}(t) in the characterization phase P2. The measurement noise then exhibits an intensity of the order of ΔSₖ^{ref}|_{Δφ} corresponding to the difference between Sₖ^{ref}|_{φ2} and Sₖ^{ref}|_{φ1}. It is then a question, in order to characterize just the compounds of interest, of correcting the measurement signal Sₖ^{Δφ}(t) for the measurement noise ΔSₖ^{ref}|_{Δφ} and of subtracting therefrom the reference value Sₖ^{ref}|_{φ1}.

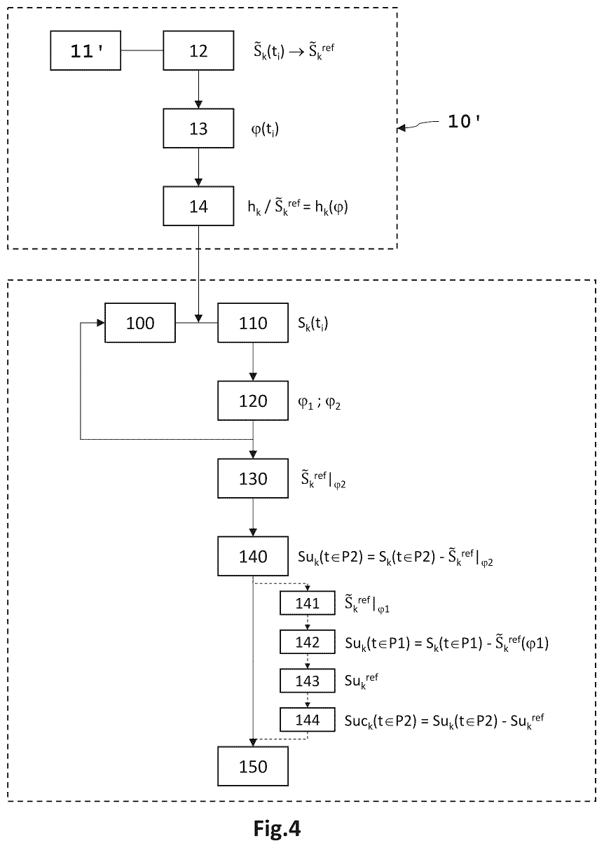

FIG. 4 illustrates a flowchart of a method for characterizing compounds of interest according to a first embodiment, in which the measurement noise due to a non-zero difference in relative humidity Δφ is decreased or even eliminated, the difference in relative humidity Δφ in the measuring chamber being defined between a value φ1 in the initial phase P1 and a value φ2, different from φ1, in the characterization phase P2. In this embodiment, the useful signal is corrected based on an estimate of a reference value \tilde{S}ₖ^{ref}|_{φ2} associated with the first gas sample for the relative humidity φ2, this estimate being obtained from a calibration function hₖ.

In this example, the measuring chamber comprises a plurality of distinct sensitive sites 6ₖ, but alternatively it may comprise only one. Additionally, the measurement signal detected by the image sensor is a portion of the excitation optical signal which is reflected by the sensitive sites 6ₖ, but alternatively it may be a transmitted portion. The electronic nose according to one embodiment comprises a humidity sensor 9 suitable for measuring the relative humidity in the measuring chamber. This humidity sensor 9 may be arranged in the measuring chamber or in the upstream or downstream fluid ducts. It is connected to the processing unit which determines the difference in relative humidity Δφ between the initial phase P1 and the characterization phase P2.

In a preliminary, calibration phase 10′, a calibration function hₖ associated with each sensitive site 6ₖ is determined. This calibration function expresses a variation in a parameter representative of the measurement signal associated with the first gas sample formed of just the carrier gas (without the compounds of interest) as a function of the relative humidity φ. More specifically, the representative parameter here is a reference value of the measurement signal when the first gas sample is present in the measuring chamber. \tilde{S}ₖ^{ref} denotes here the estimated reference value of the measurement signal \tilde{S}ₖ(t) determined in the calibration phase. More specifically, the tilde sign is placed on the letter S when the measurement signal and its reference value are associated with the calibration function. The calibration function is a continuous function which may be polynomial, logarithmic, etc. It is parameterized in calibration phase 10′, as detailed below.

In a first step 100 (also referred to herein as a “fluid injection step” 100), the step of injecting fluid into the measuring chamber of the electronic nose is performed. This step comprises a first, initial phase P1 of injecting the first gas sample (carrier gas without the compounds of interest), a second, characterization phase P2 in which the second gas sample is injected (carrier gas with the compounds of interest), and then a third, dissociation phase P3. The two gas samples exhibit different relative humidities, denoted φ1 for the first gas sample and φ2 for the second.

In a step 110, there is determined for each sensitive site 6ₖ ranging from 1 to N, at the current time a measurement signal Sₖ(t) representative of the reflectivity % Rₖ(tᵢ) of the sensitive site 6ₖ in question and, therefore, also representative of the response of the electronic nose in the presence of one and then the other of the gas samples in the measuring chamber.

For this, in fluid injection step 100, a plurality of “elementary images” Ieₘ of N sensitive sites 6ₖ are acquired. More specifically, the sensitive sites 6ₖ are illuminated by an excitation optical signal capable of generating surface plasmons thereon, and the reflected portion of the excitation optical signal is detected. The image sensor 8 is connected to the processing unit, which stores the acquired images.

The image sensor 8 acquires, over a duration Δt separating two successive measurement times tᵢ₋₁ and tᵢ, a plurality of images Ieₘ, called elementary images, of the matrix array of N distinct sensitive sites, m being the acquisition rank of the elementary image Ie, at a sampling frequency fₑ. The sampling frequency fₑ may be 10 images per second, and the acquisition period Δt may be a few seconds, 4 seconds for example.

For each elementary image Ieₘ, the processing unit determines an elementary optical intensity value (Iₖ)ₘ by taking the average of the optical intensity (Iₖ(i,j))ₘ acquired by each pixel i, j associated with a given sensitive site 6ₖ, and computes an average value (Īₖ)_{Δt} thereof over the duration of acquisition Δt. This average value (Īₖ)_{Δt} then corresponds to the measurement signal Sₖ(tᵢ), at the current time tᵢ, associated with the sensitive site 6ₖ.

This step 110 of acquiring and determining the measurement signals Sₖ(tᵢ) is carried out in the fluid injection step 100, and reiterated for multiple successive measurement times tᵢ. With each iteration i is associated one measurement time tᵢ, also called the current time.

In a step 120, the relative humidity in the measuring chamber is measured in phases P1 and P2. For this, the humidity sensor 9 measures the relative humidity φ in phases P1 and P2 and transmits the measured values to the processing unit. The relative humidity φ2 is here different from the value φ1. The relative humidity values φ1 and φ2 may be an average value of the relative humidity over this phase in question or over a given duration. The relative humidity φ1 is preferably an average value calculated over a duration directly preceding time to and, therefore, the characterization phase P2. The relative humidity φ2 is preferably an average value calculated over a duration situated in the steady state P2.2, for example over a duration directly preceding time t_{d} and, therefore, the dissociation phase P3.

In a step 130, there is determined a reference value \tilde{S}ₖ^{ref}|_{φ2} representative of the measurement signal associated with the first gas sample for a relative humidity φ2. This reference value \tilde{S}ₖ^{ref}|_{φ2} is calculated based on the calibration function Ilk and based on the measured value φ2. In other words: \tilde{S}ₖ^{ref}|_{φ2}=hₖ(φ2).

In a step 140, the measurement signal Sₖ(tᵢ) associated with the second gas sample, i.e., for tᵢ belonging to phase P2, is corrected by subtracting the determined reference value \tilde{S}ₖ^{ref}|_{φ2}. A useful signal Suₖ(tᵢϵP2) is thus obtained. Thus, in the context of this embodiment, the useful signal associated with the second gas sample, i.e., that comprising the compounds of interest but having experienced a variation in relative humidity, is calculated by correcting its measurement signal Sₖ(tᵢϵP2) in phase P2 by the reference value \tilde{S}ₖ^{ref}|_{φ2} originating from the calibration function hₖ and not by subtracting its reference value Sₖ^{ref} therefrom. Specifically, this value Sₖ^{ref} is associated with the relative humidity φ1 of the first gas sample in phase P1 while the measurement signal of the second gas sample has experienced the measurement noise due to the difference Δφ. Thus, subtracting the reference value Sₖ^{ref} from the measurement signal Sₖ(tᵢϵP2) does not allow the measurement noise to be accounted for. However, subtracting the reference value \tilde{S}ₖ^{ref}|_{φ2} originating from the calibration function hₖ therefrom accounts for the difference in relative humidity Δφ.

In a step 150, the compounds of interest are characterized based on the corrected useful signals Suₖ(tᵢϵP2). An equilibrium, i.e., steady, value is extracted from these signals in order to provide a representation in the form of a histogram, a radar chart, etc., forming the signature of the compounds of interest.

Thus, the characterization method according to this embodiment makes it possible to improve the quality of the characterization of the compounds of interest by limiting or even eliminating the measurement noise due to a variation in the relative humidity between the phases P1 and P2. The useful signal Suₖ(tᵢϵP2) allowing the compounds of interest to be characterized is calculated by correcting the measurement signal Sₖ(tᵢϵP2) associated with the second gas sample by a reference value \tilde{S}ₖ^{ref}|_{φ2} associated with the first gas sample and with the relative humidity φ2. This amounts to estimating the reference value \tilde{S}ₖ^{ref}|_{φ2} that the first gas sample would have for the relative humidity φ2, and then subtracting this value from the measurement signal Sₖ(tᵢ) detected in step 110. The characterization of the compounds of interest is then made more accurate and precise insofar as it relates to just the compounds of interest and not, or only slightly, to the carrier gas which has experienced a variation in its relative humidity.

Steps 141 to 144 may be carried out in an advantageous manner. They make it possible to further improve the quality of the characterization of the compounds of interest in the case where the electronic nose exhibits sensor drift, that is to say a variation in the measurement signal issued by the electronic nose even though the compounds of interest and the operating conditions are the same. This sensor drift occurs here between calibration phase 10′ and characterization phase 100-150.

In step 141, there is determined the reference value \tilde{S}ₖ^{ref}|_{φ1} representative of the measurement signal associated with the first gas sample for a relative humidity φ1. This reference value \tilde{S}ₖ^{ref}|_{φ1} is calculated based on the calibration function hₖ and based on the measured value φ1.

In other words, \tilde{S}ₖ^{ref}|_{φ1}=hₖ(φ1).

In step 142, the measurement signal Sₖ(tᵢ) associated with the first gas sample, i.e., for tᵢ belonging to phase P1, is corrected by subtracting the determined reference value \tilde{S}ₖ^{ref}|_{φ1}. Thus obtained is a useful signal Suₖ(tᵢϵP1), such that Suₖ(tᵢϵP1)=Sₖ(tᵢ)−\tilde{S}ₖ^{ref}|_{φ1}. Due to the sensor drift, the useful signal Suₖ(tᵢϵP1) is not substantially zero while it should be.

In step 143, the reference value Suₖ^{ref} of the useful signal Suₖ(tᵢϵP1) is determined. This is, for example, the average value of this useful signal over a predefined duration, before time to and, therefore, before phase P2.

In step 144, the useful signal Suₖ(tᵢϵP2) associated with the second gas sample is corrected by subtracting therefrom the determined reference value Suₖ^{ref}. A corrected useful signal Sucₖ(tᵢϵP2) which ignores the sensor drift is thus obtained.

In step 150, the compounds of interest are characterized based on the corrected useful signal Sucₖ(tᵢϵP2). Insofar as the sensor drift is corrected, a characterization of the compounds of interest of improved quality is obtained.

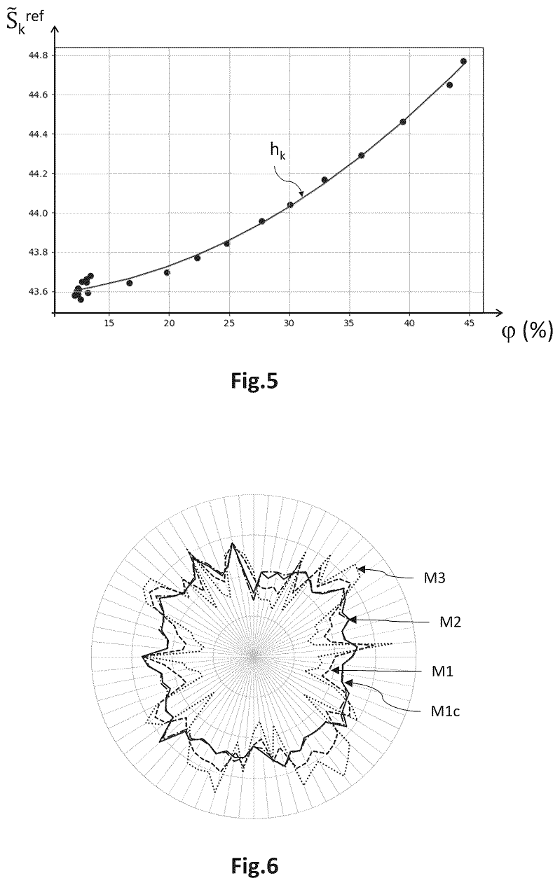

The calibration phase 10′ is now described with reference to FIG. 4 and FIG. 5, which illustrates an example of variation in the reference value S associated with a first gas sample as a function of the relative humidity φ. In this phase, the tilde sign is used on the letter S to differentiate the measurement signals acquired in this phase from those acquired in the characterization phase.

In a step 11′, the first gas sample is injected into the measuring chamber. The first gas sample is, therefore, formed of the carrier gas but does not contain the compounds of interest. It has a non-zero relative humidity φ, which varies over time, preferably in stages.

In a step 12, there is determined, for each sensitive site 6ₖ ranging from 1 to N, at the current time tᵢ, a measurement signal \tilde{S}ₖ(tᵢ) representative here of the reflectivity % Rₖ(tᵢ) of the sensitive site 6ₖ in question and, therefore, also representative of the response of the electronic nose in the presence of the first gas sample in the measuring chamber. This step is similar to step 120 and is, therefore, not described again. Insofar as the first gas sample does not contain any compounds of interest, the measurement signal \tilde{S}ₖ(tᵢ) does not exhibit the transient assimilation state P2.1 and the steady equilibrium state P2.2. It is, therefore, possible to determine a reference value \tilde{S}ₖ^{ref}|_{φ} associated with a given relative humidity value φ. It is preferably an average value of \tilde{S}ₖ(tᵢ) over a predefined duration, where φ is preferably constant.

In step 13, the relative humidity φ(tᵢ) is measured over time using the humidity sensor 9.

In step 14, the calibration function hₖ is determined based on the determined reference values \tilde{S}ₖ^{ref}|_{φ} and measured relative humidity values φ. FIG. 5 illustrates an example of a calibration function hₖ which illustrates the variation in the reference value \tilde{S}ₖ^{ref} associated with the first gas sample (humid air, for example) as a function of the relative humidity. In this example, the calibration function is a polynomial function whose parameterization, i.e., the determination of the order n of the polynomial and of the coefficients, is performed here by way of polynomial regression. Other types of calibration functions may be used, such as logarithmic functions, sigmoid neural networks, Gaussian mixtures, etc. Thus, following the calibration phase, there is obtained a calibration function hₖ associated with each sensitive site 6ₖ which makes it possible to determine the reference value \tilde{S}ₖ^{ref}|_{φ} associated with the first gas sample (humid air, for example) for a given relative humidity φ.

FIG. 6 illustrates the three signatures M1, M2 and M3 shown in FIG. 3A, The signature M1c corresponds to the same second gas sample as for the signature M1, i.e., a gas sample formed of humid air with a relative humidity φ2 equal to approximately 50% (therefore, with a variation in relative humidity Δφ) and whose compounds of interest are butanol molecules. While the signature M1 is obtained using a characterization method according to an example from the prior art, the signature M1c is obtained using the characterization method illustrated in FIG. 4. It is noted that the signature M1c is superimposed over the signature M2 which corresponds to an absence of variation in relative humidity Δφ between phases P1 and P2. Thus, the characterization method according to this embodiment of the disclosure effectively makes it possible to reduce or even eliminate the measurement noise associated with a non-zero variation in relative humidity between phases P1 and P2.

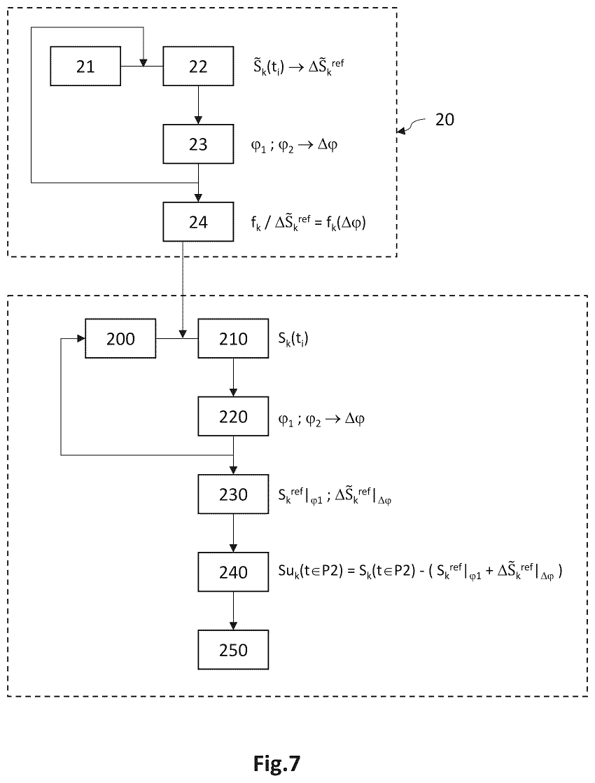

FIG. 7 illustrates a flowchart of a method for characterizing compounds of interest according to a second embodiment, in which the measurement noise due to a non-zero difference in relative humidity Δφ between phases P1 and P2 is decreased or even eliminated, the difference in relative humidity Δφ in the measuring chamber being defined between a value φ1 in the initial phase P1 and a value φ2, different from φ1, in the characterization phase P2. This method differs from that described in FIG. 4 primarily in that the useful signal is corrected using, in particular, an estimate of the difference in reference value \tilde{S}ₖ^{ref}|_{Δφ} obtained from a calibration function fₖ.

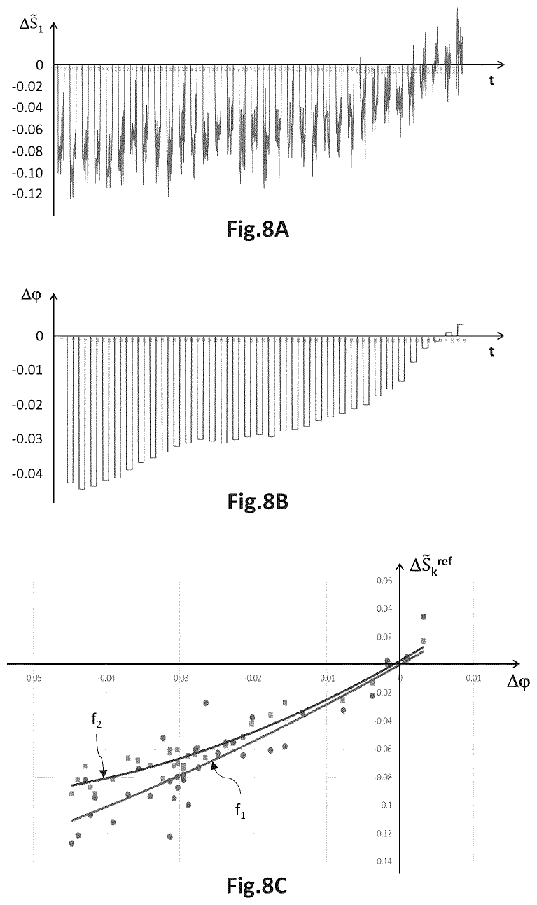

In a calibration phase 20, the calibration function fₖ is determined. This phase is illustrated in FIGS. 8A to 8C. The objective is to determine a calibration function fₖ expressing the variation in the difference in reference value \tilde{S}ₖ^{ref}|_{Δφ} as a function of a difference in relative humidity Δφ.

In a step 21, multiple injection cycles of injecting the first gas sample into the measuring chamber are carried out. The first gas sample is, therefore, formed of the carrier gas but does not contain the compounds of interest. Each cycle is formed of a first injection of the first gas sample at a constant and non-zero relative humidity φ1, followed by a second injection of the first gas sample at a constant relative humidity φ2 different from φ1. Over the various cycles, the relative humidity φ2 varies, such that a plurality of values of the difference in relative humidity Δφ=φ2−φ1 are obtained.

In a step 22, there is acquired the measurement signal \tilde{S}ₖ(tᵢ) in step 21. FIG. 8A illustrates more precisely the difference ΔŚ₁(tᵢ) between the measurement signals associated with the two injections of each cycle for a sensitive site 6₁ as a function of time. More specifically, the measurement signal of the first injection is subtracted from the measurement signal of the second injection. Thus, as shown in FIG. 8A, the difference ΔŚ₁(tᵢ) has a zero value in the first injections and a non-zero value in the second injections.

The difference in the reference values \tilde{S}ₖ^{ref} between the first and second injections of each cycle is then determined. This means determining the reference value \tilde{S}ₖ^{ref} for the first and second injections of each cycle and determining the difference. The reference value \tilde{S}ₖ^{ref} is preferably an average value of the measurement signal \tilde{S}ₖ(tᵢ) over a predefined period.

In a step 23, the difference in relative humidity Δφ between the first and second injections of each cycle is measured. FIG. 8B illustrates the variation in the difference in relative humidity Δφ as a function of time. It is noted that the difference Δφ is zero for each first injection, and that it is non-zero and varies over time from one second injection to the next.

In a step 24, the calibration function fₖ is determined based on the determined values of the difference in reference value \tilde{S}ₖ^{ref} and measured values of the difference in relative humidity Δφ0 FIG. 7C illustrates an example of two calibration functions f₁, f₂ which illustrates the variation in the difference in reference value \tilde{S}ₖ^{ref} associated with the first gas sample (e.g., humid air) as a function of the difference in relative humidity Δφ. In this example, the calibration function is a polynomial function whose parameterization, i.e., the determination of the order n of the polynomial and of the coefficients, is performed here by way of polynomial regression. Other types of calibration functions may be used, such as logarithmic functions, sigmoid neural networks, Gaussian mixtures, etc., like for the embodiment of FIG. 4. Thus, following the calibration phase, there is obtained a calibration function fₖ associated with each sensitive site 6ₖ which makes it possible to determine the difference in reference value \tilde{S}ₖ^{ref} associated with the first gas sample (e.g., humid air) for a given difference in relative humidity Δφ.

Next, calibration phase 200 to 250 is carried out. In a step 200, the step of injecting fluid into the measuring chamber of the electronic nose is performed. This step comprises a first, initial phase P1 of injecting the first gas sample (carrier gas without the compounds of interest), a second, characterization phase P2 in which the second gas sample is injected (carrier gas with the compounds of interest), and then a third, dissociation phase P3. The two gas samples exhibit different relative humidities, denoted φ1 for the first gas sample and φ2 for the second.

In a step 210, there is determined for each sensitive site 6ₖ ranging from 1 to N, at the current time tᵢ, a measurement signal Sₖ(tᵢ) representative of the response of the electronic nose in the presence of one and then the other of the gas samples in the measuring chamber. This step is similar to step 110 and is not described in detail again.

In a step 220, the relative humidity in the measuring chamber is measured in phases P1 and P2. For this, the humidity sensor 9 measures the relative humidity φ in phases P1 and P2 and transmits the measured values to the processing unit. The relative humidity φ2 is here different from the value φ1. The processing unit then determines the difference in relative humidity Δφ=φ2−φ1.

In a step 230, there is determined the estimate of the difference in reference value \tilde{S}ₖ^{ref}|^{Δφ} based on the measured difference in relative humidity Δφ. This difference in reference value \tilde{S}ₖ^{ref}|_{Δφ}, as shown in FIG. 3B, corresponds to the effect on the measurement signal associated with the second gas sample caused by the difference in relative humidity Δφ between phases P1 and P2. This difference in reference value \tilde{S}ₖ^{ref}|_{Δφ} is calculated from the calibration function fₖ and from the measured difference Δφ. Next, there is also determined the reference value \tilde{S}ₖ^{ref}|_{φ1} associated with the first gas sample based on the measurement signal Sₖ(tᵢϵP1). This is preferably an average value of the measurement signal Sₖ(tᵢϵP1) in phase P1 over a predefined duration.

In a step 240, the measurement signal Sₖ(tᵢ) associated with the second gas sample, i.e., for tᵢ belonging to phase P2, is corrected by subtracting the estimate of the difference in reference value \tilde{S}ₖ^{ref}|_{Δφ} for the measured difference in relative humidity Δφ, and by subtracting the reference value \tilde{S}ₖ^{ref}|_{φ1} associated with the first gas sample. A useful signal Suₖ(tᵢϵP2) is thus obtained. In other words, Suₖ(tᵢϵP2)=Sₖ(tᵢϵP2)−[Δ\tilde{S}ₖ^{ref}|_{Δφ}+Sₖ^{ref}|_{φ1}]. Thus, in the context of this embodiment, the useful signal associated with the second gas sample, i.e., that comprising the compounds of interest but having experienced a variation in relative humidity, is calculated by correcting its measurement signal Sₖ(tᵢϵP2) by subtracting therefrom both the effect \tilde{S}ₖ^{ref}|_{Δφ} caused by the difference in relative humidity Δφ and the reference value Sₖ^{ref}|_{φ1}. Thus, this embodiment makes it possible to exclude the measurement noise associated with the difference in relative humidity Δφ, and also to exclude any sensor drift.

In a step 250, the compounds of interest are characterized based on the useful signals Suₖ(tᵢϵP2). An equilibrium, i.e., steady, value is extracted from these signals in order to provide a representation in the form of a histogram, a radar chart, etc., forming the signature of the compounds of interest.

Thus, the characterization method according to this embodiment not only makes it possible to improve the quality of the characterization of the compounds of interest by limiting or even eliminating the measurement noise due to a variation in the relative humidity between the phases P1 and P2, but also by limiting or even eliminating any sensor drift between the calibration phase and the characterization phase. The characterization of the compounds of interest is then made more accurate and precise insofar as it relates to just the compounds of interest and not, or only slightly, to the carrier gas which has experienced a variation in its relative humidity.

Particular embodiments have just been described. Various modifications and variants will be obvious to anyone skilled in the art.

1. A method for characterizing compounds of interest introduced into a measuring chamber of an electronic nose comprising at least one sensitive site having receptors with which the compounds of interest are able to interact through adsorption/desorption, the method comprising:
injecting, into the measuring chamber:
in a first phase P1, a first gas sample formed of a carrier gas without the compounds of interest; and
in a second phase P2 following the first phase P1, a second gas sample formed of at least the carrier gas and of the compounds of interest;
determining, in the first and second phases P1 and P2, a measurement signal (Sₖ(tᵢ)) representative of interactions between the gas sample present and the receptors of the sensitive site (6ₖ), at various measurement times tᵢ, in response to an excitation signal issued at the sensitive site (6ₖ);
measuring:
a value φ1 of relative humidity φ in the measuring chamber in the first phase P1, and
a value φ2 of relative humidity in the second phase P2, the value of relative humidity in the second phase φ2 being different from the value of relative humidity in the first phase φ1;
determining a corrective parameter (\tilde{S}ₖ^{ref}|_{φ2}; Δ\tilde{S}ₖ^{ref}|_{Δφ}) associated with the sensitive site (6ₖ), based on:
at least the measured value φ2 of the relative humidity in the second phase, and
a predetermined calibration function (fₖ, hₖ) associated with the sensitive site (6ₖ), the predetermined calibration function (fₖ, hₖ) expressing a variation in a parameter (\tilde{S}ₖ^{ref}; Δ\tilde{S}ₖ^{ref}) representative of the measurement signal associated with the first gas sample as a function of the relative humidity (φ; Δφ); and
determining a useful signal (Suₖ(tᵢϵP2)) by correcting the measurement signal (Sₖ(tᵢϵP2)) associated with the second gas sample based on at least the determined corrective parameter (\tilde{S}ₖ^{ref}|_{φ2}; Δ\tilde{S}ₖ^{ref}|_{Δφ}), and characterizing the compounds of interest based on the useful signal (Suₖ(tᵢϵP2)).

2. The method of claim 1, further comprising a phase of determining the calibration function denoted by hₖ comprising:
injecting the first gas sample into the measuring chamber such that the relative humidity φ varies progressively, and measuring the relative humidity φ;
in the injecting, determining a measurement signal \tilde{S}ₖ(tᵢ), and then determining a reference value \tilde{S}ₖ^{ref} based on the determined measurement signal \tilde{S}ₖ(tᵢ) and for each measured relative humidity value φ; and
determining the calibration function hₖ expressing the variation in the reference value \tilde{S}ₖ^{ref} as a function of the relative humidity φ, based on the determined reference values \tilde{S}ₖ^{ref} and the measured relative humidity values φ.

3. The method of claim 2, wherein the corrective parameter is a reference value \tilde{S}ₖ^{ref}|_{φ2} representative of the measurement signal Sₖ(tᵢ) of the first gas sample for the measured relative humidity φ2.

4. The method of claim 2, wherein:
determining the useful signal comprises:
determining a reference value \tilde{S}ₖ^{ref}|_{φ1} representative of the measurement signal of the first gas sample for the measured relative humidity φ1, based on the calibration function hₖ;
determining a useful signal Suₖ(tᵢϵP1) associated with the first gas sample by subtracting the determined reference value \tilde{S}ₖ^{ref}|_{φ1} from the measurement signal Sₖ(tᵢϵP1) determined in the first phase P1;
determining a reference value Suₖ^{ref} based on the determined useful signal Suₖ(tᵢϵP1) associated with the first gas sample; and
determining a corrected useful signal Sucₖ(tᵢϵP2) associated with the second gas sample by subtracting the determined reference value Suₖ^{ref} from the useful signal Suₖ(tᵢϵP2); and
after determining the corrected useful signal, characterizing the compounds of interest based on the corrected useful signal Sucₖ(tᵢϵP2) associated with the second gas sample.

5. The method of claim 1, further comprising a phase of determining the calibration function denoted by fₖ comprising:
injecting the first gas sample into the measuring chamber such that multiple injection cycles are carried out, each cycle comprising a first injection of the first gas sample at a relative humidity φ1 followed by a second injection of the first gas sample at a relative humidity 2 different from φ1, and determining the difference in relative humidity Δφ between φ1 and φ2 varying from one cycle to the next;
determining a measurement signal \tilde{S}ₖ(tᵢ) in the various multiple injection cycles, and determining a difference in reference values Δ\tilde{S}ₖ^{ref} for each injection cycle and for each determined difference in relative humidity Δφ, based on the measurement signal Śₖ(tᵢ) determined in the multiple injection cycles; and
determining the calibration function fₖ expressing the variation in the difference in reference value Δ\tilde{S}ₖ^{ref} as a function of the difference in relative humidity Δφ, based on the determined values of the difference in reference value Δ\tilde{S}ₖ^{ref} and the determined values of the difference in relative humidity Δφ.

6. The method of claim 5, wherein the corrective parameter is the sum of:
a value of the difference in reference values Δ\tilde{S}ₖ^{ref} for the difference in relative humidity Δφ determined based on the relative humidity measured in the first phase P1 and in the second phase P2, and
a reference value Sₖ^{ref}|_{φ1} associated with the first gas sample and determined based on the measurement signal Sₖ(tᵢϵP1) determined in the first phase P1.

7. The method of claim 6, wherein determining the useful signal comprises subtracting the corrective parameter from the measurement signal Sₖ(tᵢϵP2) associated with the second gas sample and determined in the second phase P2.

8. The method of claim 1, wherein the predetermined calibration function (fₖ; hₖ) is a polynomial, logarithmic, or exponential function.

9. The method of claim 1, wherein the predetermined calibration function (fₖ; hₖ) is a second-degree polynomial function.

10. The method of claim 1, wherein the electronic nose comprises a device for measuring the interactions between the compounds of interest and the surface plasmon resonance optical receptors.

11. The method of claim 1, wherein the electronic nose comprises a device for measuring the interactions between the compounds of interest and the resistive, piezoelectric, mechanical, acoustic or optical receptors.
